(19) United States
(12) Patent Application Publication
(10) Pub. No.: US 2021/0077284 A1
(43) Pub. Date: Mar. 18, 2021

(54) SCAFFOLD FOR VASCULAR PROTHESIS AND A METHOD OF FABRICATING THEREOF

(71) Applicant: NANYANG TECHNOLOGICAL UNIVERSITY, Singapore (SG)

(72) Inventors: Subramanian VENKATRAMAN, Singapore (SG); Jean-Marc Benedikt BEHR, Singapore (SG); Scott Alexander IRVINE, Singapore (SG)

(73) Assignee: NANYANG TECHNOLOGICAL UNIVERSITY, Singapore (SG)

(21) Appl. No.: 16/965,914

(22) PCT Filed: Jan. 30, 2019
(86) PCT No.: PCT/SG2019/050047

§ 371 Date: Jul. 29, 2020

(30) Foreign Application Priority Data
Jan. 30, 2018 (SG) 10201800813Q

(51) Int. Cl. A61F 2/89; A61F 2/07; A61L 31/06; A61L 31/14

(52) U.S. Cl. CPC A61F 2/89; A61F 2/07; A61F 2240/001; A61L 31/14; A61F 2230/0004; A61L 31/06

(57) ABSTRACT

A scaffold for vascular prosthesis of blood vessel may include an inner tube made of elastomeric material, and an outer mesh which surrounds the inner tube and which is made of a material of a higher stiffness than the elastomeric material of the inner tube. The outer mesh may include a plurality of coils wound around the inner tube. Each of said coils may be parallel to each other. The outer mesh may further include at least one linking strand connecting two or more said coils to each other. Each of said coils may include one or more axially-oriented-kinks.

### CROSS-REFERENCE TO RELATED APPLICATIONS

The present application claims the benefit of the Singapore patent application No. 10201800813Q filed on 30 Jan. 2018, the entire contents of which are incorporated herein by reference for all purposes.

### TECHNICAL FIELD

Various embodiments generally relate to a scaffold for vascular prosthesis and a method of fabricating a scaffold for vascular prosthesis. In particular, various embodiments generally relate to a scaffold for vascular prosthesis suitable to be used in small diameter blood vessel and a method of fabricating a scaffold vascular prosthesis suitable to be used in small diameter blood vessel.

### BACKGROUND

Small diameter vascular grafts fail shortly after implantation due to blockages developing in the graft. A primary reason for this is the mismatch between the mechanical behaviour of the native artery and that of its replacement. This is usually termed “compliance mismatch”. At today's state of the art, saphenous vein grafts remain as the golden standard. However, many patients lack healthy tissue that can be used as appropriate replacement. Large diameter vessel grafts made out of polyethylene terephthalate (PET) and polytetrafluoroethylene (PTFE) have been used for decades. Unfortunately, for smaller diameters (<6 mm) there is still no suitable blood vessel prosthesis that works well. The most common reasons of failure after in vivo implantation include:
Compliance mismatch at the anastomosis site, which later will cause neo-intimal hyperplasia that later ends up in graft occlusion; and/or
Thrombosis, due to incompatibility between the grafting material and host's blood.

To date, neither any tissue-engineered construct, nor any vascular prosthesis made out of synthetic materials has been approved for small-diameter blood vessel grafting. Based on studies, the requirements that make a small diameter blood vessel prosthesis work successfully in vivo are:
Biocompatibility of the grafting material, i.e. causing no inflammation and not being immuno- or thrombogenic; and/or
The prosthesis needs to have elastic properties that match with native arteries, regarding compliance, visco-elasticity and non-linear elastic stress-strain response (J-Curve behavior), which are of great importance for small diameter artery grafting.

Some other requirements that are not critical, but may have a beneficial effect are:
Easy handling, for example for suturing;
Exhibiting high kink resistance;
Easy fabrication process that is not too time consuming, versatile and suitable for producing grafts with various diameters; and/or
Easy sterilization.

Further, past and recent studies that deal with the issue of compliance matching, mainly try to target a single compliance value (at physiological range 80-120 mm Hg) and do not consider the non-linear elastic nature of native arteries, whereby due to the structure of the arterial walls, native blood vessels tend to become stiffer at higher pressures.

Accordingly, there is a need for a scaffold for vascular prosthesis, in particular for vascular prosthesis of small blood vessel, and a method of fabricating thereof.

### SUMMARY

According to various embodiments, there is provided a scaffold for vascular prosthesis of blood vessel. The scaffold may include an inner tube made of elastomeric material, and an outer mesh which surrounds the inner tube and which is made of a material of a higher stiffness than the elastomeric material of the inner tube. The outer mesh may include a plurality of coils wound around the inner tube. Each of said coils may be parallel to each other. The outer mesh may further include at least one linking strand connecting two or more said coils to each other. Each of said coils may include one or more axially-oriented-kinks.

According to various embodiments, there is provided a method of fabricating a scaffold vascular prosthesis of blood vessel. The method may include providing an inner tube made of elastomeric material, forming an outer mesh in a two-dimensional form, wherein the outer mesh is made of a material of a higher stiffness than the elastomeric material of the inner tube, and arranging the outer mesh around the inner tube so as to surround the inner tube. The outer mesh may include a plurality of coils wound around the inner tube, each of said coils being parallel to each other. The outer mesh may further include at least one linking strand connecting at least two of said coils to each other. Each of said coils may include one or more axially-oriented-kinks.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments are described with reference to the following drawings, in which:

FIG. 1A (extracted from ‘Groenink, M., et al., The influence of aging and aortic stiffness on permanent dilation and breaking stress of the thoracic descending aorta. Cardiovascular research, 1999. 43(2): p. 471-480’) shows the non-linear elastic stress-strain curve of a blood vessel;

FIG. 1B (extracted from Tai, N., et al., Compliance properties of conduits used in vascular reconstruction. British Journal of Surgery, 2000. 87(110: p. 1516-1524′) shows how the non-linear elastic nature of the artery impacts the compliance, compared to commonly used materials that have only a single elastic modulus;

FIG. 2 shows a scaffold for vascular prosthesis of blood vessel according to various embodiments;

FIG. 3A shows various types of fibers with different geometries for use in a scaffold according to various embodiments;

FIG. 3B shows fibers with different step-length to height ratios, LHR, for use in a scaffold according to various embodiments;

FIG. 3C shows various examples of ZZ fibers with different segment lengths for use in a scaffold according to various embodiments;

FIG. 3D shows various examples of HEX-fibers with different number of kinks for use in a scaffold according to various embodiments;

FIG. 3E, FIG. 3F, and FIG. 3G show more types of of fibers with different geometries for use in a scaffold according to various embodiments;

FIG. 3H shows that a kink of a fiber may have either rounded corners or sharp corners according to various embodiments;

FIG. 4 shows different type of bridges for linking the respective fibers according to various embodiments;

FIG. 5A shows an outer mesh of a scaffold according to various embodiments;

FIG. 5B shows an outer mesh, which is similar to the outer mesh 220a of FIG. 5A but of double length for two layered wrapping (2R) around an inner tube, according to various embodiments;

FIG. 5C shows a section of the pattern of the outer mesh of FIG. 5B on the inner tube of a scaffold according to various embodiments;

FIG. 5D, FIG. 5E, and FIG. 5F shows various configuration for an outer mesh of a scaffold according to various embodiments;

FIG. 6A shows a method of fabricating a scaffold according to various embodiments;

FIG. 6B shows a photograph of three actual scaffolds fabricated by the method of FIG. 6A, wherein each scaffold is sitting on a metallic rod, according to various embodiments;

FIG. 7 and FIG. 8 show various method of fabricating a scaffold 200 according to various embodiments;

FIG. 9A shows a light microscopy image at 2× magnification of a ZZ-fiber outer mesh according to various embodiments;

FIG. 9B shows a light microscopy image at 2× magnification of a HEX-fiber according to various embodiments;

FIG. 10 shows 0.1-Curves for the HEX-fibers during tensile testing according to various embodiments;

FIG. 11A and FIG. 11B show the same scaffold according to various embodiments with FIG. 11A showing the scaffold before high stress is applied and FIG. 11B showing the fibers are nearly fully stretched out;

FIG. 12 illustrates the compliance of the scaffold, obtained from averaging the curves of three specimens, according to various embodiments;

FIG. 13 shows a singular specimen graph for FIG. 12 according to various embodiments; and

FIG. 14 shows the compliance of the scaffold with HEX-fiber of different LHR according to various embodiments.

### DETAILED DESCRIPTION

Embodiments described below in the context of the apparatus are analogously valid for the respective methods, and vice versa. Furthermore, it will be understood that the embodiments described below may be combined, for example, a part of one embodiment may be combined with a part of another embodiment.

It should be understood that the terms “on”, “over”, “top”, “bottom”, “down”, “side”, “back”, “left”, “right”, “front”, “lateral”, “side”, “up”, “down” etc., when used in the following description are used for convenience and to aid understanding of relative positions or directions, and not intended to limit the orientation of any device, or structure or any part of any device or structure. In addition, the singular terms “a”, “an”, and “the” include plural references unless context clearly indicates otherwise. Similarly, the word “or” is intended to include “and” unless the context clearly indicates otherwise.

Various embodiments generally relate to a scaffold for vascular prosthesis of blood vessel and a method of fabricating a scaffold for vascular prosthesis of blood vessel. In particular, various embodiments are targeted at small diameter blood vessels. According to various embodiments, the scaffold may be a tissue-engineered construct or a vascular prosthesis made out of synthetic materials, or an artificial blood vessel, or a synthetic vascular graft.

Various embodiments may be a combination of a soft highly compliant inner tube with a wavy bio-printed outer mesh such that the construct provides a non-linear elastic behaviour mimicking a blood vessel. The bio-printed outer mesh (or “J-Mesh”) may have tunable mechanical properties by altering the dimensions of the elastic struts, in order to match the stiffening response of native small diameter arteries.

According to various embodiments, there is provided a new scaffold or construct that may be configured as follows. The scaffold (or the tubular construct) may be made of Poly-L-lactide-co-caprolactone (PLC, with lactide to caprolactone ratios ranging from 50:50 to 95:5), or bio-elastomeric poly-caprolactone-glycolide (PCG 35:65-50:50), or poly-caprolactone (PCL), or poly-L-lactide (PLLA), or polyurethane (PU), or thermoplastic polyurethane (TPU) or other suitable elastomeric material. The scaffold for vascular prosthesis may include two components: a soft inner tube made out of elastomeric materials; and a stiffer wavy outer stent-like bioprinted mesh made of stiff materials. The mesh may be made of elastomeric materials that are stiffer than the elastomeric materials of the inner tube.

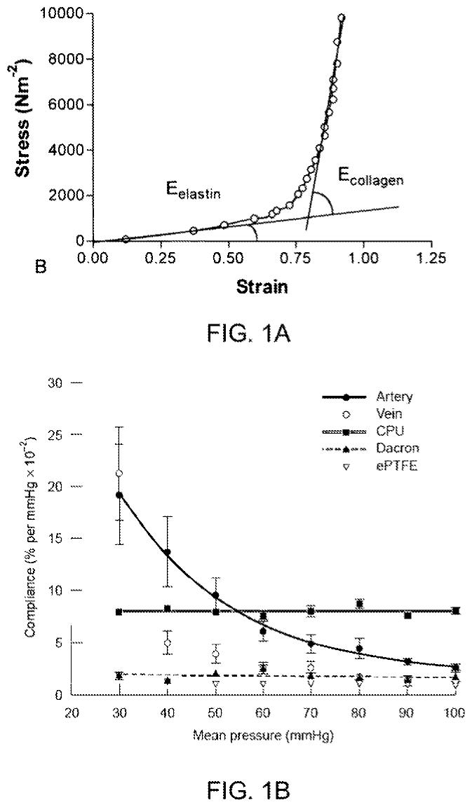

According to various embodiments, the inner tube (or the tubular graft) may be fabricated via dip-coating and may be configured to mimic the elastin-rich media layer of a blood vessel, while the bio-printed outer mesh may represent the tunica adventitia of the blood vessel. In the blood vessel, the wavy collagen fibers in the adventitia layer have the ability to straighten out at low stress, and only get stretched at higher stresses once they are fully unfolded. Further, the elastic modulus of the collagen fiber exceeds the modulus of the elastin from the media layer, which at low pressures may be the mechanically dominant structure. According to various embodiments, the scaffold may be configured to behave similarly to the blood vessel. FIG. 1A shows a stress-strain diagram of an artery with the characteristic J-Curve, indicating a non-linear elastic behaviour. FIG. 1B shows compliance decay curve of arteries, showing a steep decrease in elasticity at low pressures. The J-Curve as seen in FIG. 1A manifests itself through an increase in elastic modulus. As compliance is inversely proportional to the elastic-modulus (stiffness), it is decreasing towards higher pressures as seen in FIG. 1B.

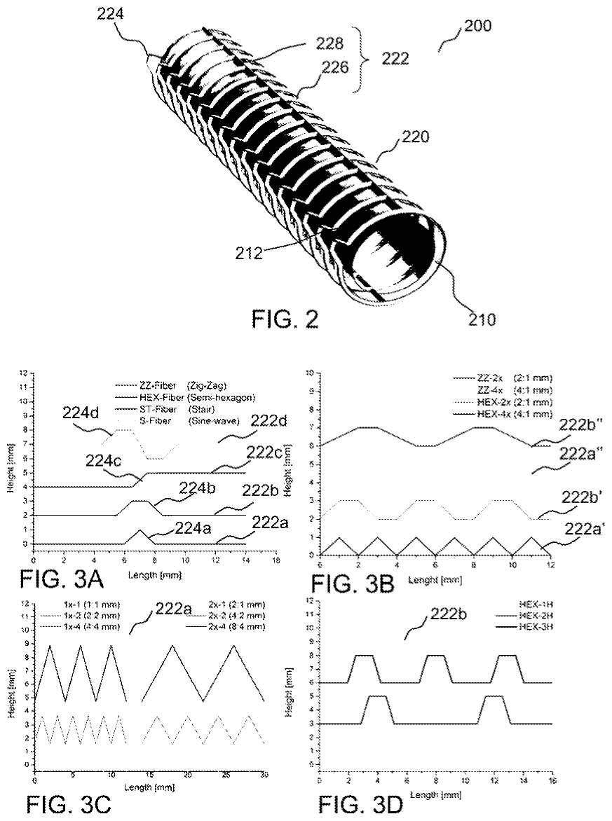

FIG. 2 shows a scaffold 200 for vascular prosthesis of blood vessel according to various embodiments. The scaffold 200 may include an inner tube 210 made of elastomeric material. The scaffold may further include an outer mesh 220 which surrounds the inner tube 210 and which is made of a material of a higher stiffness than the elastomeric material of the inner tube 210. According to various embodiments, the elastomeric material of the inner tube 210 may include elastomeric copolymer. The elastomeric copolymer may include poly-L-lactide-co-caprolactone (PLC), or bio-elastomeric poly-caprolactone-glycolide (PCG), or poly-caprolactone (PCL), or poly-L-lactide (PLLA), or poly-lactide (PLA), or polyurethane (PU), or thermoplastic polyurethane (TPU), or other suitable elastomeric copolymer.

According to various embodiments, the inner tube 210 may be a hollow cylindrical tube resembling a section of a blood vessel. According to various embodiments, a diameter of the inner tube 210 may be less than 6 mm, or less than 5 mm. Accordingly, the inner tube 210 may resemble a small diameter blood vessel.

According to various embodiments, the outer mesh 220 may be a stent-like mesh including wavy fibers 222 (or strands) that may unfold at low modulus and later upon complete straightening induce a stiff response (similar to a J-Curve response). According to various embodiments, the outer mesh 220 may be fabricated using a 3D printing robot.

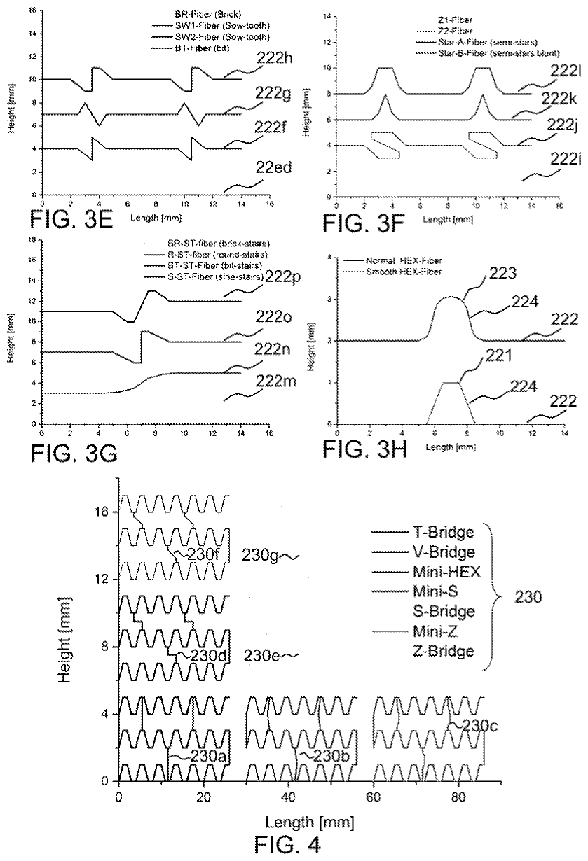

According to various embodiments, the fibers 222 (or strands) of the outer mesh 220 may be configured with wavy features by including one or more kinks 224 (or springs) that may unfold in a spring-like manner. More examples of the fibers 222 (or strands) are shown in FIG. 3A. Accordingly, the one or more kinks 224 may initially unfold as the respective fiber 222 (or strand) is stretched under tension. After the respective fiber 222 (or strand) is straightened, further application of tension may cause the respective fiber 222 (or strand) to elastically deform and stretch. Subsequently, the respective fiber 222 (or strand) may return to the original shape with the folded kink when the tension force is removed. According to various embodiments, the fibers 222 (or strands) with wavy features may form a plurality of coils 226 wound around the inner tube 210. According to various embodiments, one coil 226 may complete one single loop or round around the circumference of the inner tube 210. According to various embodiments, each of the plurality of coils 226 may be parallel to each other. According to various embodiments, each of the plurality of coils 226 may be a closed coil or an opened coil. According to various embodiments, each of the plurality of coils 226 may be a circumferential coil or a helical coil.

According to various embodiments, each of the one or more kinks 224 of the respective coil 226 may be an axially-oriented kink 224 such that the respective kink 224 may be parallel to a circumferential surface 212 of the inner tube 210. Accordingly, the respective kink 224 may be directed in an axial direction of the respective coil 226. In this manner, during radial expansion of the inner tube 210, the respective coil 226 may be subjected to a radial expansion force which may stretch the respective coil 226 circumferentially in a manner such that the axially-oriented kink 224 of the respective coil 226 may unfold at low stress and, upon complete straightening, may require higher stress to further stretch and deform the fully unfolded coil 226. Hence, the scaffold 200 may exhibit a non-linear elastic behaviour resembling the J-Curve when such an expansion force is applied.

According to various embodiments, the axially-oriented-kinks 224 of axially adjacent ones of the plurality of coils 226 may axially engage with each other. Accordingly, the axially-oriented-kinks 224 of axially adjacent ones of the plurality of coils 226 may be arranged such that the axially-oriented-kinks 224 of subsequent coil 226 may be received within the axially-oriented-kinks 224 of the earlier coil in a manner similar to stacking such that the axially-oriented-kinks 224 of axially adjacent ones of the plurality of coils 226 may be stacked one after another in sequence. According to various embodiments, the axially-oriented kinks 224 of all of the plurality of coils 226 may be oriented in the same axial direction. Accordingly, the axially-oriented kinks 224 of all of the plurality of coils 226 may have the same orientation.

According to various embodiments, the fibers 222 (or strands) of the outer mesh 220 may include at least one linking strand 228 (or linking fiber) connecting two or more coils 226 to each other. According to various embodiments, the at least one linking strand 228 (or linking fiber) may extend in a longitudinal direction with respect to the inner tube 210. According to various embodiments, the linking strands 228 (or the linking fibers) may intersect the plurality of coils 226 to form the outer mesh 220 such that the linking strands 228 (or linking fibers) and the plurality of coils 226 may join in such a way so as to form the outer mesh 220 as a single unit or a complete structural whole. According to various embodiments, the longitudinally extending linking strand 228 (or linking fiber) may also include wavy features, i.e. the one or more kinks 224.

According to various embodiments, the at least one linking strand 228 may be disposed in between two kinks 224 of one of the plurality of coils 226. Accordingly, the at least one linking strand 228 may intersect the coil 226 at a segment of the coil 226 between the two kinks 224 of the one of the plurality of coils 226.

According to various embodiments, the fibers 222 (or strands) of the outer mesh 220 with the wavy features (or the spring-like fibers) may be capable of inducing a J-curve stress-strain response. Hence, these fibers 222 (or strands) may be named as “J-Fibers”. FIG. 3A shows various types of fibers 222a, 222b, 222c, 222d with different geometries. As shown, fiber 222a may be a zig-zag fiber (ZZ-fiber) having a kink 224a in the form of a zig-zag which resemble a pointed peak or a triangle or an-inverted V shape. Fiber 222b may be a semi-hexagon fiber (HEX-fiber) having a kink 224b in the form of a semi-hexagon (or half of a hexagon or three sides of a hexagon). Fiber 222c may be a stair fiber (ST-fiber) having a kink 224c in the form of a slant inclination such that a segment of the fiber 222c before the kink 224c and a segment of the fiber 222c after the kink 224c are at different levels or elevation. Fiber 222d may be a sine-wave fiber (S-fiber) having a kink 224d resembling a crest and a trough of a sine-wave. The crest and the trough may be a smooth curve or may be straight lines. According to various embodiments, a profile of the kink 224 may be classified into a single bend profile, a double bend profile, or a step profile. According to various embodiments, the single bend profile may include a kink 224 that is formed by bending the fiber 222 (or strand) upon itself once to form into a crest-like or a trough-like formation. Accordingly, the zig-zag kink 224a of the fiber 224a of FIG. 3A and the semi-hexagon kink 224b of the fiber 224b of FIG. 3A may be examples of the single bend profile. According to various embodiments, the double bend profile may include a kink 224 that is formed by bending the fiber 222 (or strand) upon itself twice to form a crest-like and a trough-like formations. Accordingly, the sine-wave-like kink 224d of the fiber 224d of FIG. 3A may be an example of the double bend profile. According to various embodiments, the step profile may include a kink 224 that is formed by bending the fiber 222 (or strand) without the fiber 222 (or strand) folding upon itself. Rather, in the step profile, a segment of the fiber 222 before the kink 224 and a segment of the fiber 222 after the kink 224 may be at different levels or elevation. Accordingly, the stair-like kink 224c of the fiber 224c of FIG. 3A may be an example of the step profile.

Referring to FIG. 3A, the ZZ-fiber (or the fiber 224a), the HEX-fiber (or the fiber 224b) and the S-fiber (or the fiber 224d) may straighten out during unfolding. However, the ST-fiber (or the fiber 224c) may unfold to a diagonal line first and later align into horizontal/circumferential direction.

After the kink 224 of the fiber 222 adopts or sticks to a particular geometry, the angle of the kink 224 (or hoop, the term ‘hoop’ is also used in the description to refer to the bend or the twist in the fiber) (for example as shown in FIG. 3B) or the number of kinks 224 (or hoops) (for example as shown in FIG. 3D) may be altered, in order to tune the point of J-Curve initiation. Decreasing the angle of the kink 224 (or hoop angle) or increasing the number of kinks 224 (or hoops) may cause the fiber 222 to unfold only at higher strains. Further, the length of the segments may be increased, see for example FIG. 3C, in order to gain more flexibility during the fiber 222 unfolding. FIG. 3B shows fibers 222a′, 222b′, 222a″, 222b″ with different step-length to height ratios, LHR, (or length to amplitude ratio, L/A-ratio) or in other words differing angle of kink 224 (or hoop angles). Increasing the LHRs indicate larger angles, making the kink 224 (of hoop) flatter which may allow the fiber 222 to unfold earlier. FIG. 3C shows various examples of ZZ fibers (the fiber 224a of FIG. 3A) with different segment lengths. FIG. 3D shows various examples of HEX-fibers (the fiber 224b of FIG. 3A) with different number of kinks (or hoops (H)). Every inclusion of kink (or hoop) may delay the unfolding as the length of the fully elongated fiber 222 increases. Thus, the number of kinks (or hoop-number (#H)) may be another tool to engineer the unfolding properties.

FIG. 3E, FIG. 3F, and FIG. 3G show more types of of fibers 222e, 222f, 222g, 222h, 222i, 222j, 222k, 222l, 222m, 222n, 222o, 222p with different geometries. As shown, fiber 222e may be a brick fiber (BR-fiber). Fiber 222f and fiber 222g may be a sow-tooth fiber (SW-fiber). Fiber 222h may be a bit fiber (BT-fiber). Fiber 222i, 222j may be fanciful Z fibers (Z1-fiber, Z2-fiber). Fiber 222k may be a semi-stars fiber (Star-A-fiber). Fiber 222l may be a semi-start blunt fiber (Star-B-fiber). Fiber 222m may be a brick-stair fiber (BR-ST fiber). Fiber 222n may be a round-stairs fiber (R-ST-fiber). Fiber 222o may be a bit-stairs fiber (BT-ST-fiber). Fiber 222p may be a sine-stairs fiber (S-ST-fiber). The respective kinks 224 of the fiber 222e (BR-fiber), the fiber 222k (Star-A-fiber) and the fiber 222l (Star-B-fiber) may be classified as a kink 224 having a single bend profile. The respective kinks 224 of the fiber 222f (SW1-fiber), the fiber 222g (SW2-fiber), the fiber 222h (BT-fiber), the fiber 222i (Z1-fiber), and the fiber 222j (Z2-fiber) may be classified as a kink 224 having a double bend profile. The respective kinks 224 of the fiber 222n (R-ST-fiber) and the fiber 222m (BR-ST-fiber) may be classified as a kink 224 having a step profile. The kink 224 of the fiber 222o (BT-ST-fiber) and the fiber 222p (S-ST-fiber) may be classified as a kink 224 having a combination of a double bend profile and a step profile. FIG. 3H shows that a kink 224 of a fiber 222 may have either rounded corners 223 or sharp corners 221. Accordingly to various embodiments, a kink 224 of a fiber 222 may also have a combination of rounded corners and sharp corners (not shown).

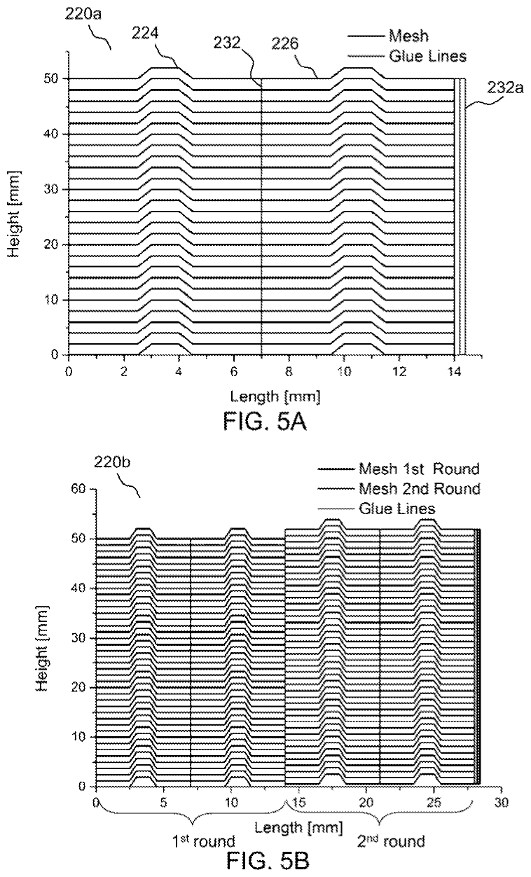

According to various embodiments, for better integrity of the fibers 222 (or strands), the fibers 222 may optionally be linked with different type of bridges (or bridge-links). FIG. 4 shows different type of bridges (or bridge-links 230) linking the respective fibers 222. According to various embodiments, the at least one linking strand 228 of the outer mesh 220 may be a bridge-link 230 between two adjacent coils of the plurality of coils 226. Accordingly, the bridge-link 230 may connect two adjacent coils of the plurality of coils 226 together. According to various embodiments, the bridge-link 230 may extend axially between the two adjacent coils of the plurality of coils 226 for joining the two adjacent coils.

As shown in FIG. 4, the bridges (or bridge-links 230) may be a T-bridge 230a, a V-bridge 230b, a mini-HEX bridge 230c, a mini-S bridge 230d, a S-bridge 230e, a mini-Z bridge 230f, or a Z-bridge 230g. According to various embodiments, each of the bridge-link 230 may have a profile including any one of a straight profile, a step profile or a bend profile. For example, the T-bridge 230a may be a straight profile. Each of the V-bridge 230b and the mini-Hex bridge 230c may be a bend profile. Each of the mini-S bridge 230d, the S-bridge 230e, the mini-Z bridge 230f, and the Z-bridge 230g may be a step profile.

According to various embodiments, the T-bridges 230a may be the only bridge that may not have any spring feature (or spring-like effect). All other types of bridges (or bridge-links 230) may work or function similar to the fibers, i.e. showing some compliance at low stresses before they straighten out. According to various embodiments, these bridges (or bridge-links 230), with the exception from T-bridges 230a, may be configured to provide longitudinal compliance when used between coils 226. According to various embodiments, the S and Z bridges 230e, 230g connect not the opposite tips but the adjacent tips, while the mini-S and the mini-Z bridges 230d, 230f connect the tip to the adjacent valley of the neighbouring fiber 222.

According to various embodiments, the outer mesh 220 may include multiple bridge links 230 with at least one bridge-link 230 between each successive pair of the plurality of coils 226. Accordingly, each of the plurality of coils 226 may be connected to the next coil via the at least one bridge link 230. Hence, the outer mesh 220 may be formed by the plurality of coils 226 and the at least one bridge-link 230 between each pair of the plurality of coils 226. According to various embodiments, the multiple bridge links 230 may be in a staggered arrangement. Accordingly, a first bridge-link 230 between a first coil of the plurality of coils 226 and a second coil of the plurality of coils 226 may be off-set circumferentially from a second bridge-link 230 between the second coil of the plurality of coils 226 and a third coil of the plurality of coils 226, for example as shown in FIG. 4.

According to various embodiments, the outer mesh 220 may be loosely abutting the inner tube 210. Accordingly, the outer mesh 220 may be free-standing with respect to the inner tube 210. Hence, the outer mesh 220 may be free of being fasten or secured or attached or fixed to the inner tube 210. Thus, the outer mesh 220 may be loosely encircling the inner tube 210 such that the outer mesh 220 may be a free-standing cage encircling the inner tube 210.

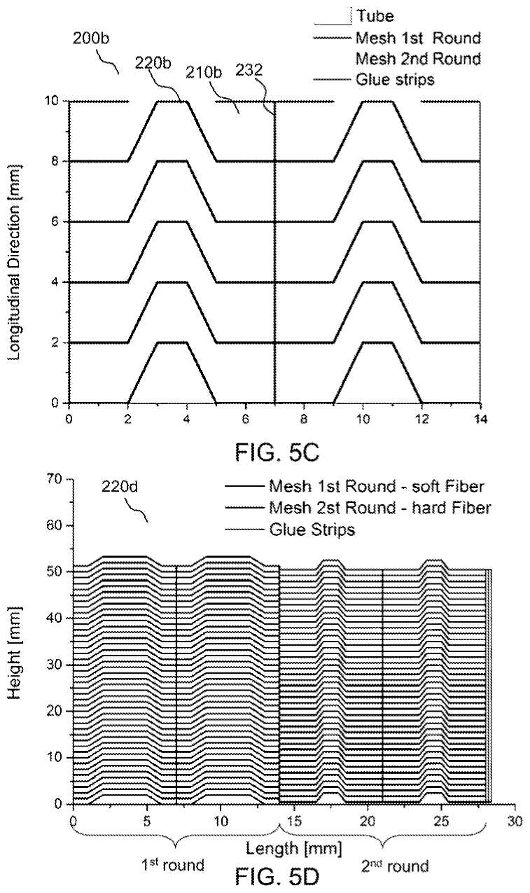

According to various embodiments, the at least one linking strand 228 may include an elongate stripe 232 (see FIG. 5A) extending longitudinally across the plurality of coils 226 so as to connect all of said coils 226 together. Accordingly, the elongate stripe 232 may intersect all the plurality of coils 226 to form the outer mesh 220. According to various embodiments, the elongate stripe 232 may be a plurality of bridge-links 230 aligned longitudinally.

According to various embodiments, when each of the plurality of coils 226 includes two kinks 224, the elongate stripe 232 may be extending longitudinally across the plurality of coils 226 between respective two kinks 224 of respective coil 226. According to various embodiments, the elongate stripe 232 may intersect the plurality of coils 226 at respective segment of the respective coil 226 between the respective two kinks 224.

According to various embodiments, the elongate stripe 232 of the outer mesh 220 may include a wavy profile. Accordingly, the elongate stripe 232 may include one or more kinks 224′ (see FIG. 5E) in a circumferential direction with respect to the inner tube 210 to form the wavy profile. According to various embodiments, the various configurations applicable to the axially-oriented kinks 224 of the plurality of coils 226 may also be applicable to the one or more kinks 224′ of the elongate stripe 232.

According to various embodiments, the outer mesh 220 may be fixed to the inner tube 210 along the elongate stripe 232 via adhesive. Accordingly, the elongate stripe 232 may serve an adhesive stripe on which adhesive may be applied such that the elongate stripe 232 of the outer mesh 220 may be stuck to the inner tube 210 as the elongate stripe 232 is being brought into contact with an exterior cylindrical surface of the inner tube 210.

According to various embodiments, the elongate stripe 232 in the form of adhesive stripe (or glue-stripe) may serve as sites for fusion to the dip-coated tubes (or the inner tube 210). The adhesive stripe (or the glue-strip) may be a center of fixation and stress-transfer from the inner tube 210 to the outer mesh 220 (or J-Mesh). According to various embodiments, the outer mesh 220 may include one or more elongate stripes 232 serving as adhesive stripes (or glue stripes). The purpose of including more adhesive stripes (or glue strips) may be for establishing a more even stress-distribution among the fibers 222. According to various embodiments, the fiber spacing may be reduced, in order to create more homogeneous stresses within the underlying inner tube 210 (or dip-coated tube). According to various embodiments, multiple wrapping of fibers 222 around the inner tube 210, and/or changing/alternating the fiber materials may be possible variation for tuning the stiff response of the outer mesh 220 (or J-Mesh).

FIG. 5A shows an outer mesh 220a (or a J-Mesh) according to various embodiments. As shown the outer mesh 220a may be a HEX-0.5x-2H mesh with three elongate stripes 232 serving as adhesive strips (or glue strips). According to various embodiments, the fibers 222 for the coils 226 may be divided into multiple segments by including elongate stripes 232 (or adhesive stripes or glue strips) between the kinks 224 (or hoops), so that every single kink 224 (or hoop) becomes an individual spring. According to various embodiments, the outer mesh 220a (or the J-Mesh) may be fused to the inner tube 210 at the elongate stripes 232. According to various embodiments, as shown in FIG. 5A, the thick stripe 232a on the right side of the outer mesh 220a may be the anchor line, where the outer mesh 220a gets fused to the inner tube 210 before the outer mesh 220a may be wrapped around the inner tube 210.

FIG. 5B shows an outer mesh 220b (or a J-Mesh) which is similar to the outer mesh 220a of FIG. 5A but of double length for two layered wrapping (2R) around the inner tube 210. Accordingly, the outer mesh 220b may be wound two rounds around the inner tube 210. In a first run of winding, a first half of the outer mesh 220b may be wound a first complete round around the inner tube 210. In a second run of winding, a second half of the outer mesh 220b may be wound a second complete round around the inner tube 210. According to various embodiments, there may be an offset between the first half of the outer mesh 220b for the first round and the second half of the outer mesh 220b for the second round in order to cover the remaining free surface of the inner tube 210 with fibers 222 from the second wrap. This may ensure denser fiber coverage for the scaffold 200 (or the JM-tube composites). FIG. 5C shows a section of the pattern of the outer mesh 220b of FIG. 5B on the inner tube 210b of the scaffold 200b (or the JM tube). According to various embodiments, the outer mesh 220 (or the J-Mesh) may be laying on the inner tube 210b and fused only at the elongate stripes 232 (or adhesive/glue strips). The first half (or the first round) of the outer mesh 220b may be shown by the black lines, while the second half (or the second round) of the outer mesh 220b may be represented by the white lines.

FIG. 5D shows an outer mesh 220d (or a J-Mesh) which is similar to the outer mesh 220b of FIG. 5B with the first half (or the first run) of the outer mesh 220d being made of a soft material and having blunt kinks (or hoops) and the second half (or the second run) of the outer mesh 220d being made of stiffer fibers that may unfold later. According to various embodiments, the outer mesh 220d of FIG. 5D may unfolds in two steps.

FIG. 5E shows an outer mesh 220e (or a Dual-J-Mesh) with longitudinally compliant elongate stripes 232 (or adhesive/glue-strips). According to various embodiments, the longitudinally compliant elongate stripes 232 may include one or more kinks 224′.

FIG. 5F shows an outer mesh 220f (or a Hel-J-Mesh) with diagonal fibers that are presented as helical fibers when wound around the inner tube 210 (or the dip-coated tube). Accordingly, the plurality of coils 226f formed may be helical coils. According to various embodiments, the fibers 222f (or J-Fibers) forming the coils 226f unfold first into straight helical lines and later align into circumferential direction.

According to various embodiments, the inner tube 210 may be fabricated via the dip-coating fabrication protocol as illustrated in the table below.

TABLE 1
Dip-coating fabrication protocol.

| Material | Concentration | # of immersions |
|---|---|---|
| PLC 50:50 | 15% | 2-3 |
| PLC 70:30 | 13.5% | 1-3 |

According to various embodiments, the outer mesh 220 may be fabricated via printing, such as 2D printing, 3D printing, or bio-printing. According to various embodiments, the outer mesh 220 may be fabricated via bio-printing with the concentrations of the polymers in chloroform as shown in table 2 below.

TABLE 2
Concentrations of the polymers in chloroform
for the fabrication of the bio-printed mesh.

| Material | Concentration |
|---|---|
| PLC 85:15 | 12% |
| PLC 95:5 | 12% |
| PCL | 12% |

According to various embodiments, the outer mesh 220 (or the J-Mesh) may be made of Poly-L-lactide-co-caprolactone (PLC), poly-caprolactone (PCL), poly-L-lactide (PLA), polyurethane, thermoplastic polyurethane, or other biocompatible polymers. According to various embodiments, the outer mesh 220 may be made of elastomeric material which may be of a higher stiffness than the elastomeric material of the inner tube 210.

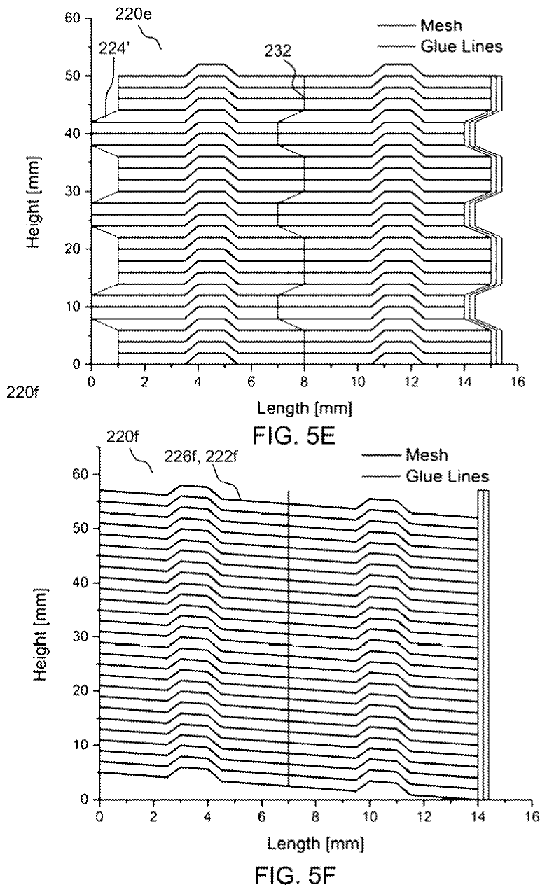

FIG. 6A shows a method 601 of fabricating a scaffold 200 (or a JM-Tube), according to various embodiments, by fusing the outer mesh 220 (or J-Mesh) at the anchor elongate stripe 232a (or the anchor glue stripe) to the inner tube 210 (or the dip-coated tube) and continuously wrapping it up. During the wrapping process, the other elongate stripes 232 (or glue-strips) may be also fused to the inner tubes 210. The result is shown on the right, which illustrate a schematic diagram of the scaffold 220. According to various embodiments, a metallic rod may be inserted through the inner tube 210 for rolling the inner tube 210 over the outer mesh 220 such that the outer mesh 220 may be wrapped around the inner tube 210. FIG. 6B shows a photograph of three actual scaffolds 200 fabricated by the method 601 with the respective scaffolds 200 sitting on respective metallic rods 603. In FIG. 6B, some fibers were coloured for better display. According to various embodiments, the outer mesh 220, such as in FIG. 5 and FIG. 6, may be printed onto glass-slides through 20-32 gauge size tips (usually 27 and 30 G) and dried in a vacuum oven overnight. In the following day, the outer mesh 220 may be fused to the inner tube 210 (or the dip-coated tubes) at the elongate stripes 232 (or the adhesive/glue-stripes), for example as shown in FIG. 6A. According to various embodiments, one line of 20 wt % glue (e.g. PLC or PLA in chloroform) may be dispensed (with a pitch of 200 μm, using a 27G needle tip as the outer mesh 220 was printed before) onto the thick anchor elongate stripe 232a (or the anchor adhesive/glue-stripe) using a 3D printer robot. According to various embodiments, a metallic mandrel with the inner tube 210 (or the dip-coated tube) may immediately be placed horizontally on the anchor elongate stripe 232a (or anchor adhesive/glue-stripe) and held for one minute, in order to allow successful solvent binding. Afterwards, the outer mesh 220 may be rolled up continuously. Similarly to the anchor elongate stripe 232a, glue may be dispensed on each other elongate stripes 232, in order to fix the outer mesh 220 at these sites to the inner tube 210. The rolling-up may complete after the sealing elongate stripe 232b (or the sealing-stripe) has been fused to the anchor elongate stripe 232a (or the anchor adhesive/glue-stripe).

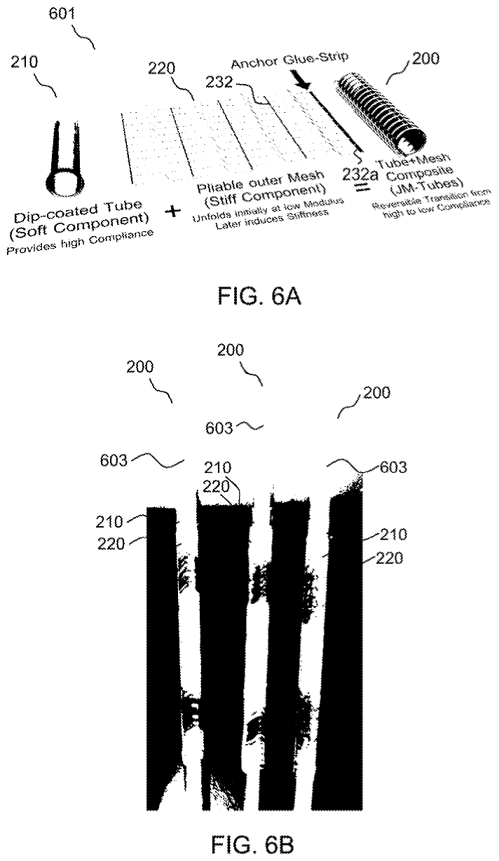

FIG. 7 shows a method 701 of fabricating a scaffold 200 (or a JM-Tube) according to various embodiments. The method 701 of FIG. 7 differs from the method 601 of FIG. 6 in that alternate to fusing the outer mesh 220 (or the J-Mesh) to the inner tube 210 (or the dip-coated tubes), the inner tube 210 (or the dip-coated tubes) of FIG. 7 are first covered with a poly(vinyl alcohol) (PVA) layer 702. The outer mesh 220 may be stuck at its first elongate stripe 232a (or the first adhesive/glue-stripe) to the PVA layer 702 and continuously wrapped up. The elongate stripes (or adhesive/glue-stripes) 232a, 232b at both ends of the outer mesh 220 may be fused through solvent binding (Polymer/solvent glue). After dissolving the PVA layer in water, the outer mesh 220 may be laying on the inner tube 210 and being a completely free-standing cage.

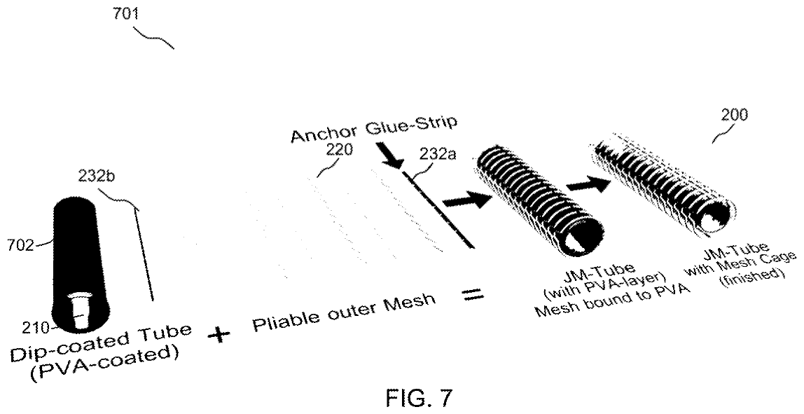

FIG. 8 shows a method 801 of fabricating a scaffold 200 (or a JM-Tube) according to various embodiments. The method 801 may be a direct fiber 222 (or J-Fiber) 3D-bioprinting onto the inner tube 210 (or the dip-coated tube). According to various embodiments, in fabricating the scaffold 200 via the method 801, the outer mesh 220 may not include any adhesive/glue-stripes. According to various embodiments, a scaffold 200 may be fabricated via direct printing of fibers 222 (or J-Fibers) onto the inner tube 210 (or the dip-coated tube). According to various embodiments, under slow rotation, the fibers 222 may be directly dispensed onto the inner tube 210 (or the dip-coated tube) by a 3D-printer. After the scaffold 220 (or the JM-Tube) composite fabrication was completed, the scaffold 220 may be dried in a vacuum oven for at least one hour and then soaked for two days in water, in order to allow the PVA layer to dissolve. Afterwards, the scaffold 220 (or the JM-tube) may be removed from their metallic mandrel and the fabrication is then completed.

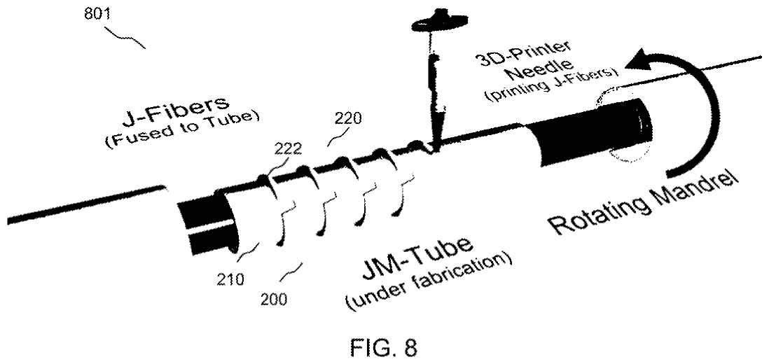

FIG. 9A shows a light microscopy image at 2× magnification of a Z7-fiber outer mesh. FIG. 9B shows a light microscopy image at 2× magnification of a HEX-fiber.

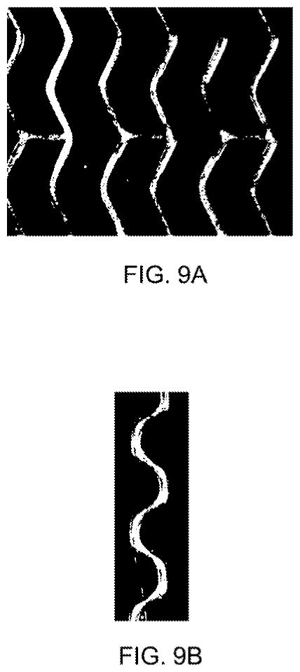

According to various embodiments, the coiled form of all fiber types may provide, like theoretically assumed, a J-shaped stress-strain curve, see FIG. 10. FIG. 10 shows J-Curves for the HEX-fibers during tensile testing. HEX-fibers with different LHR were tested. As shown, all fiber types with wavy features provided J-Curves responses, indicating the use of wavy fibers is suitable for generating a non-linear elastic behaviour. This proves that these types of fibers are suitable to induce similarly to the collagen fibers from the adventitial layers an elastic stress-strain relationship with increasing modulus. According to various embodiments, a control over the J-Curve may be achieved from altering the LHR (angle) of the kinks (or the hoops), where low LHRs, i.e. steeper angles, cause a delay in the unfolding of the fiber (full unfolding achieved at large strains).

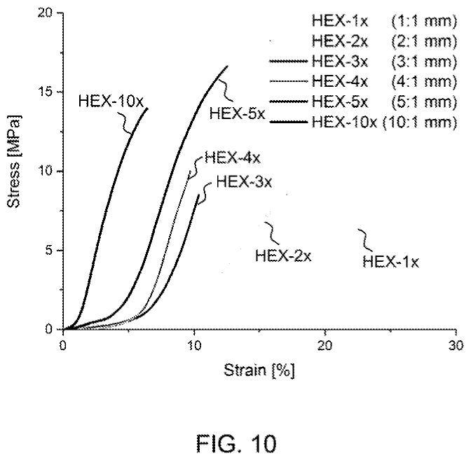

According to various embodiments, the 2D properties of the fibers that unfold during tensile testing may be successfully translated into 3D, after fusing the outer mesh (or the J-Mesh) to the inner tube (or the dip-coated tube). FIG. 11A and FIG. 11B show the same scaffold (or JM-Tube) with coloured fibers for better display. FIG. 11A shows the scaffold (or JM-Tube) before high stress is applied. As shown, the fibers are still coiled and their morphology is like in FIG. 2. FIG. 11B shows the fibers are nearly fully stretched out, indicating the unfolding of the fibers also happens after circumferential winding. According to various embodiments, at elevated internal pressure, diametric expansion may induce fiber unfolding to straight lines. After the stress got released from the scaffold (or the JM-tube) with the completely unfolded fibers, their conformation returned into a state that is identical with in FIG. 11A. This showed that the shape memory does not get lost and that strain recovery is guaranteed.

According to various embodiments, the outer mesh (or the J-Mesh) may be pre-optimized to match the arterial compliance curve over the physiological pressure range. Furthermore, experiments (with PLC 70:30 inner tube+PCL outer mesh (or J-Mesh)) have shown that the scaffold (or the JM-tube) may be capable of withstanding high luminal pressures up to 500 mm Hg. In contrast, a control of mesh-less tube (or a mesh-less scaffold or a scaffold without the outer mesh) undergoes fatigue and failure at 175 mm Hg. This underlines the protective character of the outer mesh (or the J-Mesh) in the scaffold (or the JM-tube) composite.

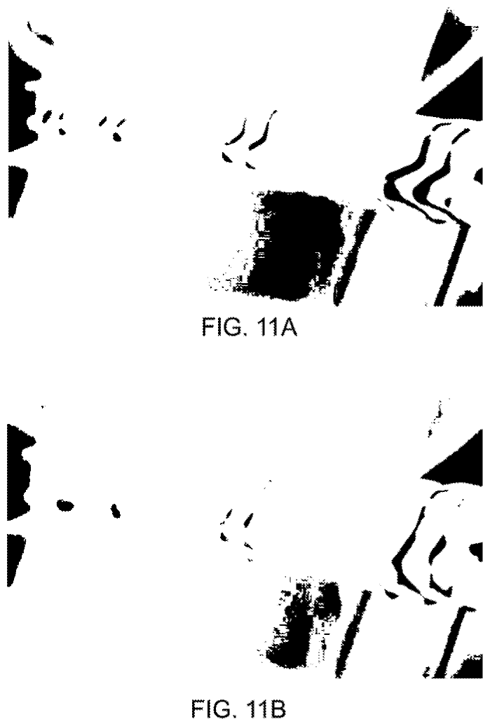

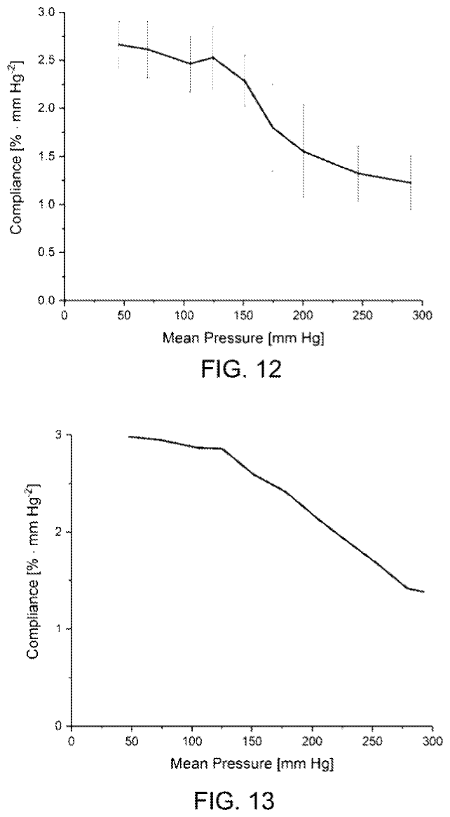

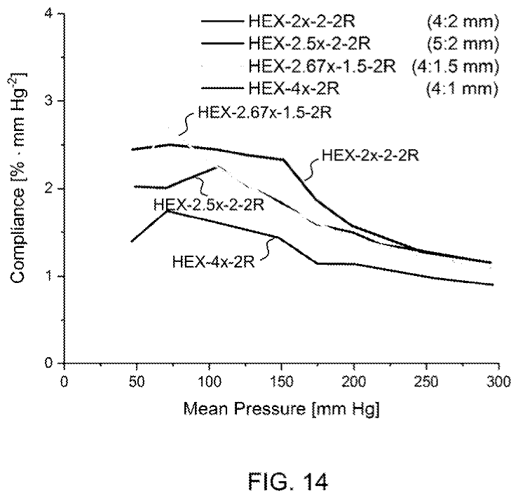

FIG. 12 illustrates the compliance of the scaffold (or the JM-Tube), obtained from averaging the curves of three specimens, showing a continuous reduction upon increasing luminal pressure. The reduction sets in at around 100 mm Hg, showing the unfolding of the outer mesh induces stiffness to the scaffold (or the JM-Tube) (n=3). Additionally, the functionality of the scaffold (or the JM-Tube) was proven during the compliance testing, showing a reduction in elasticity (radial expansion, compliance) upon increasing inner pressure. FIG. 13 shows a singular specimen graph for FIG. 12. FIG. 14 shows the compliance of the scaffold with HEX-fiber of different LHR. As shown, scaffold with different configuration may affect the respective compliance, which underline the functionality of the respective scaffold (JM-tubes).

Based on experimental results, various embodiments using Hex-fiber (see fiber 222b of FIG. 3A) with two kinks (or hoops) around a circumference of the inner tube (see FIG. 5A to FIG. 5F) may provide a preferable J-Curve stress-strain response. Thus, according to various embodiments, there is provided a scaffold for vascular prosthesis of blood vessel including an inner tube made of elastomeric material, and an outer mesh which surrounds the inner tube and which is made of a material of a higher stiffness than the elastomeric material of the inner tube. The outer mesh may include a plurality of coils wound around the inner tube, each of said coils being parallel to each other. The outer mesh may further include at least one linking strand connecting two or more said coils to each other. Further, each of said coils may include two axially-oriented kinks. Each of the two axially-oriented kinks may be of a semi-hexagonal shape (or half a hexagon). According to various embodiments, each of said coils may complete a single loop or round around the circumference of the inner tube. According to various embodiments, the two axially-oriented kinks of each coil may be disposed on opposite sides of the inner tube such that they are equidistant apart.

According to various embodiments, there is provided a scaffold for vascular prosthesis of blood vessel. The scaffold may include a tubular construct (or an inner tube) made of elastomeric copolymer. The scaffold may further include a patterned outer mesh providing non-linear elastic behaviour mimicking a blood vessel. Advantageously, the scaffold may be used for vascular prosthesis of small diameter (for example, <6 mm) blood vessel.

According to various embodiments, the elastomeric copolymer may be Poly-L-lactide-co-caprolactone (PLC, with lactide to caprolactone ratios ranging from 50:50 to 95:5) or bio-elastomeric poly-caprolactone-glycolide (PCG 35:65-50:50), or poly-caprolactone (PCL) or poly-L-lactide (PLLA), or polyurethane (PU), or thermoplastic polyurethane (TPU), or other suitable elastomeric copolymer.

According to various embodiments, the patterned outer mesh may be zig-zag (ZZ), semi-hexagon (HEX), stair (ST) and sine-wave (S) fiber configuration or any combination thereof.

Advantageously, factors such as the angle at the kinks (or hoops) as well as the number of kinks (or hoops) may be adjusted to tune the point of J-curve initiation.

According to various embodiments, there is provided a method of fabricating the scaffold. The method may include printing the patterned outer mesh in 2D on glass-slides through 20-32 gauge size tips; drying the outer mesh in a vacuum oven overnight; fusing the outer mesh to the inner tube (or the dip-coated tube) at an elongate stripe (or adhesive/glue-stripe); placing the metallic mandrel with the inner tube (or the dip-coated tube) horizontally on the anchor elongate stripe (or the anchor adhesive/glue-stripe) and hold for one minute, in order to allow successful solvent binding; and rolling the outer mesh around the inner tube (or the dip-coated tube).

According to various embodiments, there is provided a use of the above mentioned scaffold for vascular prosthesis of blood vessel.

According to various embodiments, the configuration of an artificial blood vessel, i.e. the scaffold (or the JM-tube) of the various embodiment, that is made by a bio-printed wavy outer mesh (or the J-Mesh) anchored at multiple elongate strips circumferentially to a soft inner tube may allow controlled expansion of the artificial blood vessel with increasing internal pressure in a biphasic manner of low stiffness followed by high stiffness.

According to various embodiments, the outer mesh (or the J-Mesh) may either be fused to the inner tube (or the dip-coated tube) at glue-stripes or being a free-standing cage (i.e. without being fused).

According to various embodiments, the configuration of an artificial blood vessel, i.e. the scaffold (or the JM-tube) of the various embodiment, that is made by direct printing of wavy patterns of fibers (forming an outer mesh) onto a soft inner tube, may exhibit high compliance at low internal pressures followed by a stiff response at higher pressures.

According to various embodiments, the configuration of the outer mesh fibers (or the J-Mesh fibers or the J-fibers) can be Zig-zag (ZZ), semi-hexagon (HEX), stair (ST) and sine-wave (S) design, or any combination of these as shown in FIG. 3A to FIG. 3H.

According to various embodiments, corners of a kink of a fiber (or J-Fiber spring corners) may be either sharp or rounded.

According to various embodiments, the outer mesh (or the J-Mesh) may be present on the inner tube as either circumferentially or helically wound fibers.

According to various embodiments, the outer mesh (or the J-Mesh) may optionally include longitudinally compliant fibers.

According to various embodiments, the outer mesh (or the J-Mesh) and the inner tube may be fabricated from bio-stable and biodegradable polymers.

According to various embodiments, the pattern of the outer mesh (or the J-Mesh pattern) may confer the low stiffness phase through the unfolding of the geometric pattern between the anchored points to the required limit of distension at which point the material stiffness of the polymer mesh may restrain further expansion of the inner tube.

According to various embodiments, the point at which the wavy fibers (or the J-Fibers) are fully unfolded and the stiff response of the outer mesh (or the J-Mesh) is initiated, may be shifted to lower or higher pressures by changing the fiber geometry.

According to various embodiments, the outer mesh (or the J-Mesh) may unfold, if the internal pressure is increased and may return into their initial shape upon pressure release (i.e. a reversible transition).

According to various embodiments, the pattern of the outer mesh (or the J-Mesh pattern) may be configured in a way that allows the scaffold (or the synthetic vascular graft or the JM-tube) to match the compliance of a native artery over the physiological pressure range.

According to various embodiments, the pattern of the outer mesh (or the J-Mesh pattern) may be configured in a way that causes a reduction in compliance towards increasing internal pressures of the scaffold (or the synthetic vascular graft or the JM-tube), similar to native arteries.

According to various embodiments, the outer mesh (or the J-Mesh) may protect the scaffold (or the vascular prosthesis or the JM-Tube) from aneurysmal dilatation, plastic deformation and reduces radial and longitudinal creep.

According to various embodiments, the mechanical properties during the unfolding of the outer mesh (or the J-Mesh) may be tunable.

According to various embodiments, the mechanical properties of the stiff response of the outer mesh (or the J-Mesh) may be tunable.

According to various embodiments, the outer mesh (or the J-Mesh) may not negatively affect the kink-resistance of the scaffold (or the vascular prosthesis or the JM-Tube).

The following examples pertain to various embodiments.

Example 1 is a scaffold for vascular prosthesis of blood vessel including:

an inner tube made of elastomeric material;

an outer mesh which surrounds the inner tube and which is made of a material of a higher stiffness than the elastomeric material of the inner tube,

wherein the outer mesh includes
a plurality of coils wound around the inner tube, each of said coils being parallel to each other, and
at least one linking strand connecting two or more said coils to each other,

wherein each of said coils includes one or more axially-oriented-kinks.

In Example 2, the subject matter of Example 1 may optionally include that the elastomeric material of the inner tube may include elastomeric copolymer, and wherein the elastomeric copolymer may include poly-L-lactide-co-caprolactone (PLC), or bio-elastomeric poly-caprolactone-glycolide (PCG), or poly-caprolactone (PCL), or poly-L-lactide (PLLA), or poly-lactide (PLA), or polyurethane (PU), or thermoplastic polyurethane (TPU).

In Example 3, the subject matter of Example 2 may optionally include that the PLC is with a lactide to caprolactone ratio ranging from 50:50 to 95:50.

In Example 4, the subject matter of Example 2 may optionally include that the PCG is with a caprolactone to glycolide ratio ranging from 35:65 to 50:50.

In Example 5, the subject matter of any one of Examples 1 to 4 may optionally include that the at least one axially-oriented-kink may have a profile including any one of a single bend profile, a double bend profile, or a step profile.

In Example 6, the subject matter of any one of Examples 1 to 5 may optionally include that the at least one linking strand may be disposed in between two axially-oriented-kinks of respective one of said coils.

In Example 7, the subject matter of any one of Examples 1 to 6 may optionally include that the at least one linking strand may include a bridge-link between two adjacent coils of said coils.

In Example 8, the subject matter of Example 7 may optionally include that the bridge-link may have a profile including any one of a straight profile, or a step profile, or a bend profile.

In Example 9, the subject matter of Example 7 or 8 may optionally include that the outer mesh may include multiple bridge links with at least one bridge-link between each successive pair of said coils, and wherein the multiple bridge links may be in a staggered arrangement.

In Example 10, the subject matter of any one of Examples 1 to 9 may optionally include that the outer mesh may be loosely abutting the inner tube.

In Example 11, the subject matter of any one of Examples 1 to 9 may optionally include that the at least one linking strand may include an elongate stripe extending longitudinally across the plurality of coils so as to connect all of said coils together.

In Example 12, the subject matter of Example 11 may optionally include that the elongate stripe may include a wavy profile.

In Example 13, the subject matter of Example 11 or 12 may optionally include that, when each of said coils includes two axially-oriented-kinks, the elongate stripe may be extending longitudinally across the plurality of coils between respective two axially-oriented-kinks of respective coil.

In Example 14, the subject matter of any one of Examples 11 to 13 may optionally include that the outer mesh may be fixed to the inner tube along the elongate stripe via adhesive.

In Example 15, the subject matter of any one of Examples 1 to 14 may optionally include that each of said coils may be a circumferential coil or a helical coil.

In Example 16, the subject matter of any one of Examples 1 to 15 may optionally include that the axially-oriented-kinks of axially adjacent ones of said coils axially may engage with each other.

In Example 17, the subject matter of any one of Examples 1 to 16 may optionally include that the axially-oriented kinks of all coils may be oriented in the same axial direction.

In Example 18, the subject matter of any one of Examples 1 to 17 may optionally include that each of said coils may include two axially-oriented kinks.

In Example 19, the subject matter of Example 18 may optionally include that each of the two axially-oriented kinks may be of a semi-hexagonal shape (or half a hexagon).

In Example 20, the subject matter of Example 18 or 19 may optionally include that the two axially-oriented kinks of each coil may be disposed on opposite sides of the inner tube such that they are equidistant apart.

Example 21 is a method of fabricating a scaffold vascular prosthesis of blood vessel according to any one of Examples 1 to 20. The method including:

providing an inner tube made of elastomeric material;

forming an outer mesh in a two-dimensional form, wherein the outer mesh is made of a material of a higher stiffness than the elastomeric material of the inner tube; and

arranging the outer mesh around the inner tube so as to surround the inner tube,

wherein the outer mesh includes
a plurality of coils wound around the inner tube, each of said coils being parallel to each other, and
at least one linking strand connecting at least two of said coils to each other,

wherein each of said coils includes one or more axially-oriented-kinks.

In Example 22, the subject matter of Example 21 may optionally include fixing the outer mesh to the inner tube along the at least one linking strand, wherein the at least one linking strand may include an elongate stripe extending longitudinally across the plurality of coils.

Various embodiments have provided a scaffold for vascular prosthesis of blood vessel which is able to induce a J-curve stress-strain response and which works well for small diameter blood vessels. Various embodiments have also provided a method for fabricating such scaffold.

While the invention has been particularly shown and described with reference to specific embodiments, it should be understood by those skilled in the art that various changes, modification, variation in form and detail may be made therein without departing from the scope of the invention as defined by the appended claims. The scope of the invention is thus indicated by the appended claims and all changes which come within the meaning and range of equivalency of the claims are therefore intended to be embraced.

1. A scaffold for vascular prosthesis of blood vessel comprising:
an inner tube made of elastomeric material; and
an outer mesh which surrounds the inner tube and which is made of a material of a higher stiffness than the elastomeric material of the inner tube,
wherein the outer mesh comprises
a plurality of coils wound around the inner tube, each of said coils being parallel to each other, and
at least one linking strand connecting two or more said coils to each other,
wherein each of said coils comprises one or more axially-oriented-kinks.

2. The scaffold as claimed in claim 1, wherein the elastomeric material of the inner tube comprises elastomeric copolymer, and wherein the elastomeric copolymer comprises poly-L-lactide-co-caprolactone (PLC), or bio-elastomeric poly-caprolactone-glycolide (PCG), or poly-caprolactone (PCL), or poly-L-lactide (PLLA), or poly-lactide (PLA), or polyurethane (PU), or thermoplastic polyurethane (TPU).

3. The scaffold as claimed in claim 2, wherein the PLC is with a lactide to caprolactone ratio ranging from 50:50 to 95:50.

4. The scaffold as claimed in claim 2, wherein the PCG is with a caprolactone to glycolide ratio ranging from 35:65 to 50:50.

5. The scaffold as claimed in claim 1, wherein the at least one axially-oriented-kink has a profile comprising any one of a single bend profile, a double bend profile, or a step profile.

6. The scaffold as claimed in claim 1, wherein the at least one linking strand is disposed in between two axially-oriented-kinks of respective one of said coils.

7. The scaffold as claimed in claim 1, wherein the at least one linking strand comprises a bridge-link between two adjacent coils of said coils.

8. The scaffold as claimed in claim 7, wherein the bridge-link has a profile comprising any one of a straight profile, or a step profile, or a bend profile.

9. The scaffold as claimed in claim 7, wherein the outer mesh comprises multiple bridge links with at least one bridge-link between each successive pair of said coils, and wherein the multiple bridge links are in a staggered arrangement.

10. The scaffold as claimed in claim 1, wherein the outer mesh is loosely abutting the inner tube.

11. The scaffold as claimed in claim 1, wherein the at least one linking strand comprises an elongate stripe extending longitudinally across the plurality of coils so as to connect all of said coils together.

12. The scaffold as claimed in claim 11, wherein the elongate stripe comprises a wavy profile.

13. The scaffold as claimed in claim 11, wherein, when each of said coils comprises two axially-oriented-kinks, the elongate stripe is extending longitudinally across the plurality of coils between respective two axially-oriented-kinks of respective coil.

14. The scaffold as claimed in claim 11, wherein the outer mesh is fixed to the inner tube along the elongate stripe via adhesive.

15. The scaffold as claimed in claim 1, wherein each of said coils is a circumferential coil or a helical coil.

16. The scaffold as claimed in claim 1, wherein the axially-oriented-kinks of axially adjacent ones of said coils axially engage with each other.

17. The scaffold as claimed in claim 1, wherein the axially-oriented kinks of all coils are oriented in the same axial direction.

18. A method of fabricating a scaffold vascular prosthesis of blood vessel according to claim 1, the method comprising:
providing an inner tube made of elastomeric material;
forming an outer mesh in a two-dimensional form, wherein the outer mesh is made of a material of a higher stiffness than the elastomeric material of the inner tube; and
arranging the outer mesh around the inner tube so as to surround the inner tube,
wherein the outer mesh comprises
a plurality of coils wound around the inner tube, each of said coils being parallel to each other, and
at least one linking strand connecting at least two of said coils to each other,
wherein each of said coils comprises one or more axially-oriented-kinks.

19. The method as claimed in claim 18, further comprising fixing the outer mesh to the inner tube along the at least one linking strand, wherein the at least one linking strand comprises an elongate stripe extending longitudinally across the plurality of coils.
